(19) United States
(12) Patent Application Publication
(10) Pub. No.: US 2019/0252061 A1
(43) Pub. Date: Aug. 15, 2019

(54) SYSTEM AND ARCHITECTURE FOR SEAMLESS WORKFLOW INTEGRATION AND ORCHESTRATION OF CLINICAL INTELLIGENCE

(71) Applicants: KONINKLIJKE PHILIPS N.V., EINDHOVEN (NL); The University of Chicago, Chicago, IL (US)

(72) Inventors: Paul Joseph Chang, Chicago, IL (US); Merlijn Sevenster, Haarlem (NL); Amir Mohammad TAHMASEBI MARAGHOOSH, Arlington, MA (US); Robbert Christiaan VAN OMMERING, Cambridge, MA (US); Aharona Shuali, Haifa (IL); Eliahu Zino, Atlit (IL)

(21) Appl. No.: 16/314,132

(22) PCT Filed: Jun. 21, 2017
(86) PCT No.: PCT/IB2017/053690

§ 371 Date: Dec. 28, 2018

Related U.S. Application Data
(60) Provisional application No. 62/355,417, filed on Jun. 28, 2016.

(51) Int. Cl. G16H 30/40; G06F 16/54; G06N 20/00

(52) U.S. Cl. CPC G16H 30/40; G06N 20/00; G06F 16/54

(57) ABSTRACT

A system and method for determining a desired action of a user reading a medical image. The system and method retrieving and displaying an image to be read by a user, receiving, via a processor, a contextual cue of the user in response to the displayed image to be read, mapping the contextual cue to a user intention via the processor, and generating an action based on the user intention via the processor.

### BACKGROUND

PACS (Picture Archiving Communications System) has revolutionized the way radiologists work by making workflow more efficient. This has allowed radiologists to read an increasing amount of studies at a faster rate. Further, improved workflow efficiency has led to faster reporting of critical findings, lower rescan rates, more uniformity in imaging scans, clearer actionable reports, enhanced patient experience, and improvements to reimbursements. PACS may offer even more benefits to radiologists.

Despite improvements in radiology workflow, the reading of an imaging exam or study for a patient may still be a difficult and time-consuming process for a radiologist. In some cases, radiologists spend substantial time finding relevant patient exams, selecting appropriate applications in an image processing application, waiting for the applications to finalize computation, recording key outcome findings (e.g., measurements), and inputting relevant information into a radiology report. Thus, there is still room for improvement.

As is well known, the detection, quantification, characterization and diagnosis of individual lesions in radiology exams is subject to substantial intra-rater variability and even more substantial inter-rater variability. These observations negatively affect the perception of Radiology as an exact science, and, more concretely, may degrade the satisfaction of referring physicians with the service of his/her Radiology department of choice. The exemplary embodiments below seek to resolve these issues.

### SUMMARY

A method for determining a desired action of a user reading a medical image. The method including retrieving and displaying an image to be read by a user, receiving, via a processor, a contextual cue of the user in response to the displayed image to be read, mapping the contextual cue to a user intention via the processor, and generating an action based on the user intention via the processor.

A system for determining a desired action of a user reading a medical image. The system including a display displaying an image to be read by a user and a processor receiving a contextual cue of the user in response to the displayed image to be read, mapping the contextual cue to a user intention via the processor, and generating an action based on the user intention via the processor.

A method for providing contextual interpretation support suggestions to a user. The method includes receiving a contextual cue from the user in response to a displayed image and normalizing sequences of the image onto a controlled nomenclature of sequence names. The method further includes registering imaging slices across the sequences of at least one of a plurality of imaging exams and providing contextual lesion interpretation support suggestions to the user.

### BRIEF DESCRIPTION

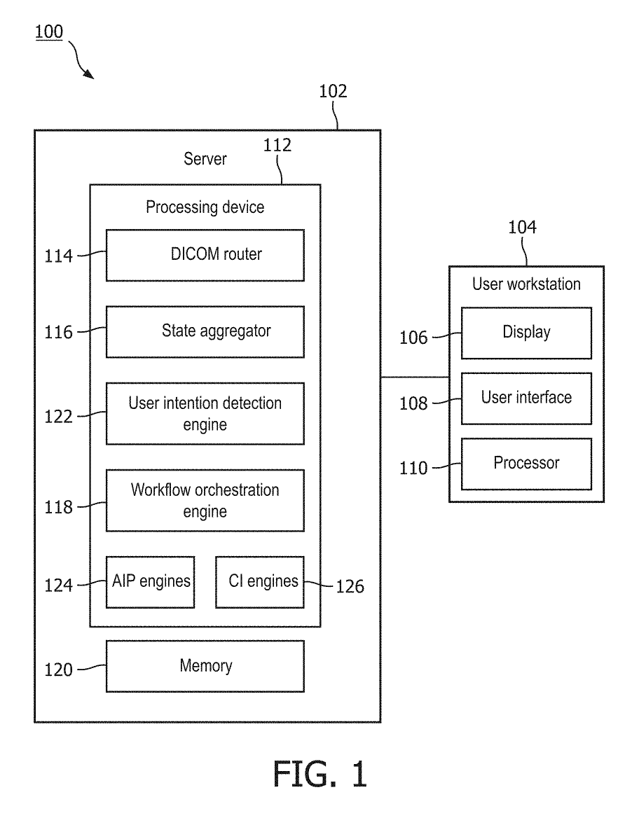

FIG. 1 shows a schematic drawing of a system according to an exemplary embodiment.

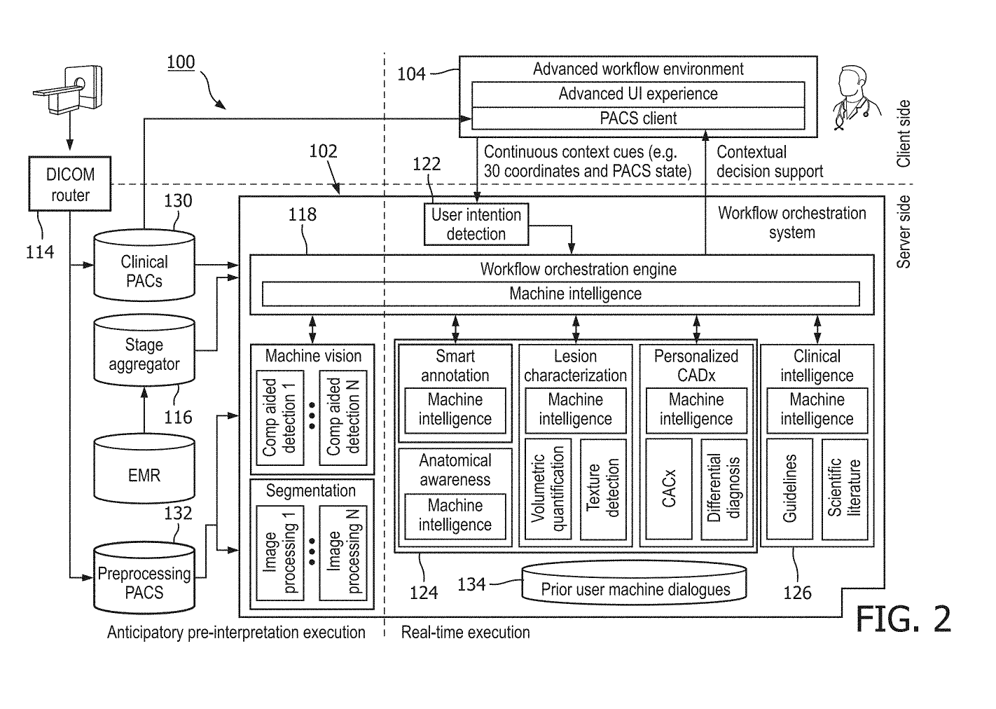

FIG. 2 shows another schematic drawing of the system of FIG. 1.

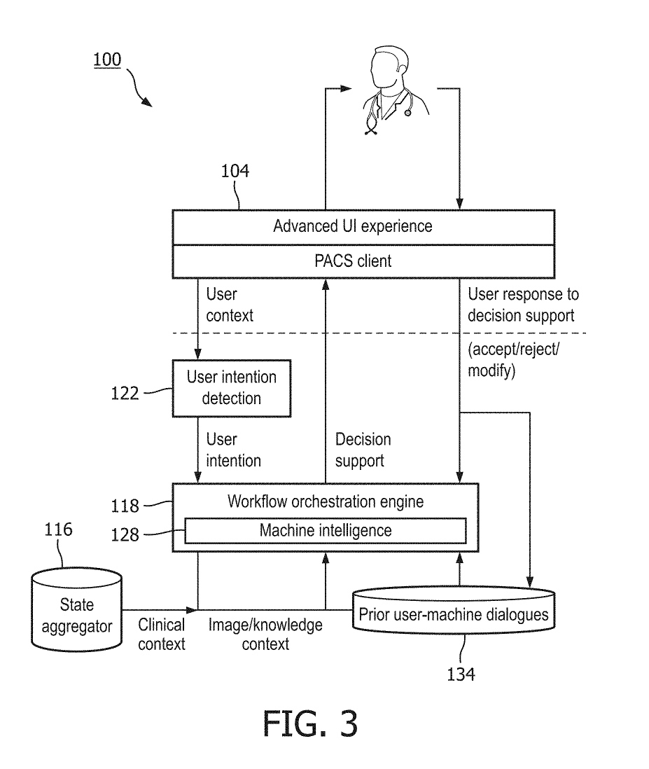

FIG. 3 shows a schematic drawing of a system according to another exemplary embodiment.

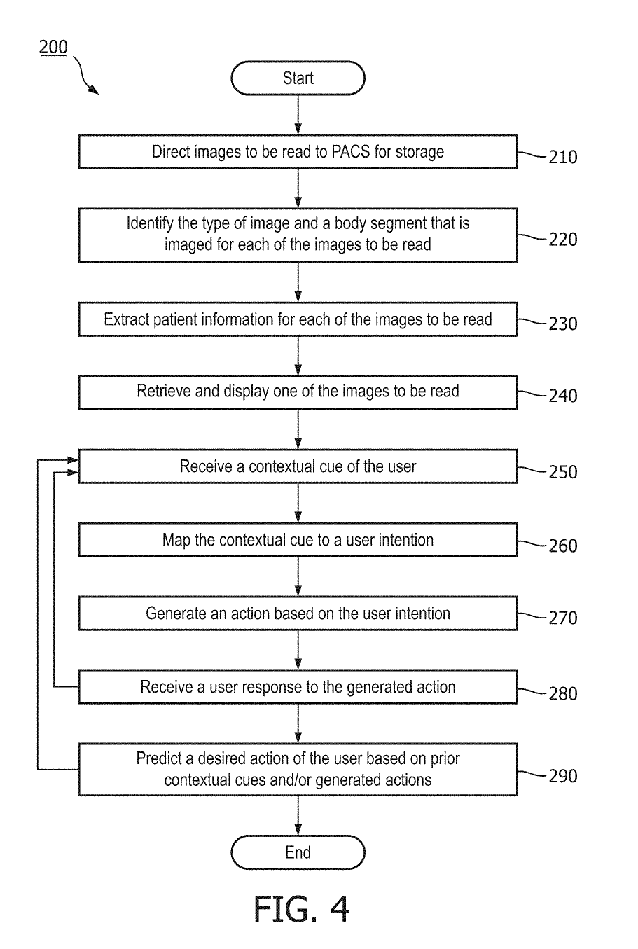

FIG. 4 shows a flow diagram of a method according to an exemplary embodiment.

FIG. 5 shows a schematic drawing of a system according to an exemplary embodiment.

FIG. 6 shows a flow diagram of a method according to an exemplary embodiment.

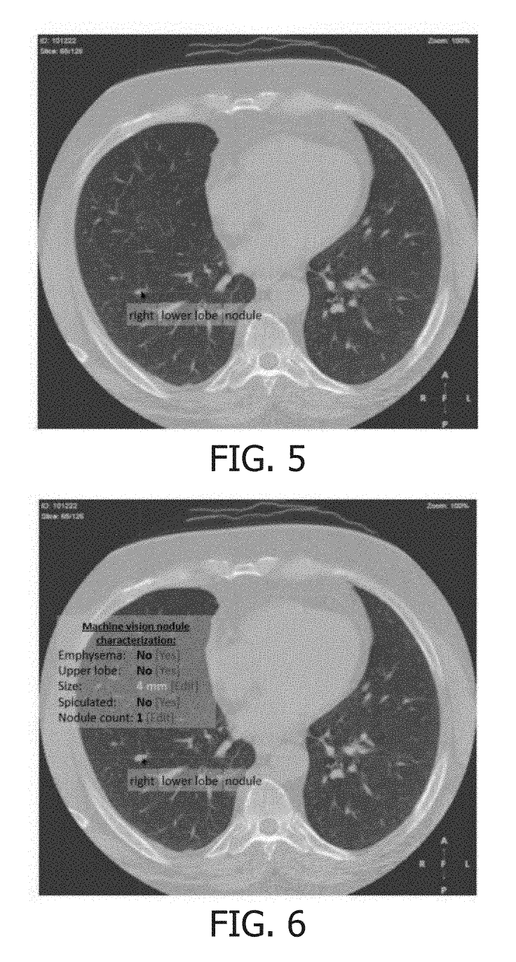

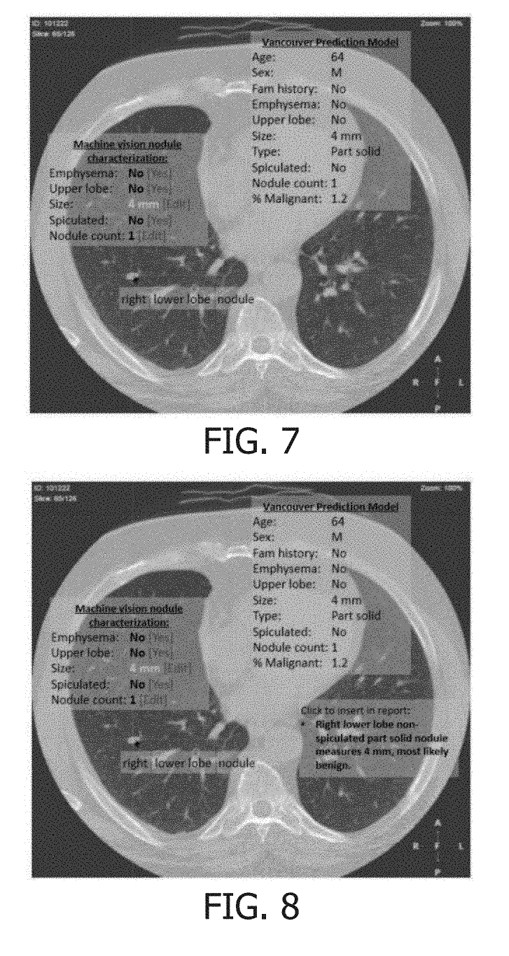

FIG. 7 shows a screenshot of a first exemplary image including lesion annotations.

FIG. 8 shows a screenshot of the first exemplary image including lesion characterizing information.

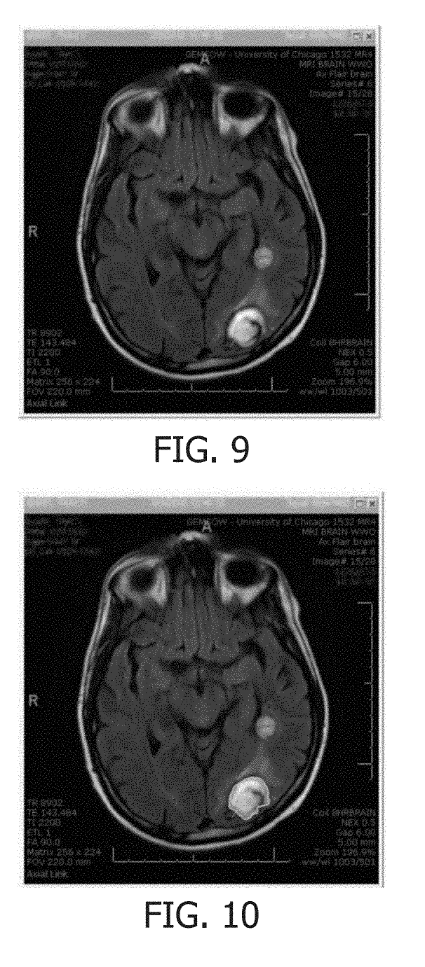

FIG. 9 shows a screenshot of the first exemplary image including a malignancy prediction for a lesion.

FIG. 10 shows a screenshot of the first exemplary image including a sentence characterizing and diagnosing the lesion.

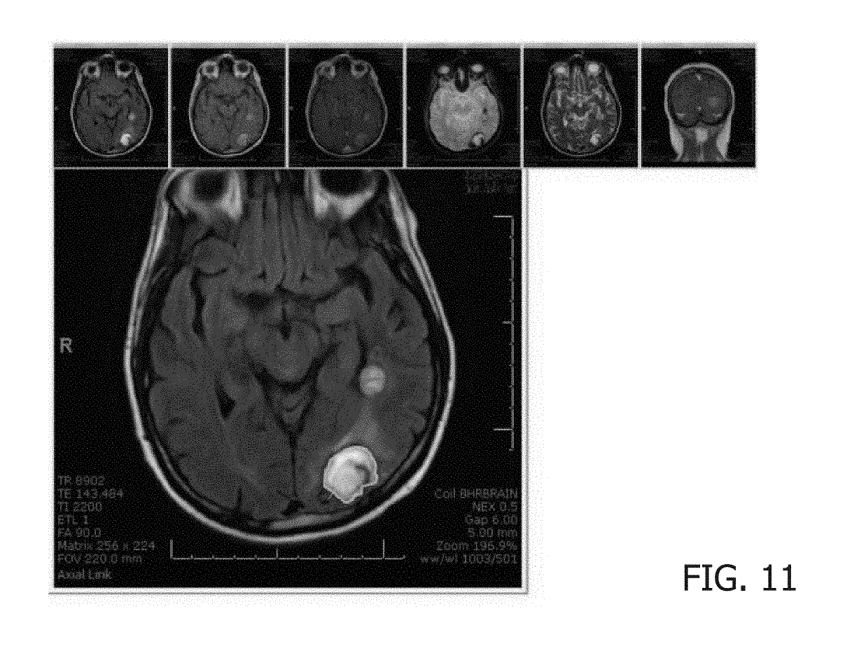

FIG. 11 shows a screenshot of a second exemplary image including a lesion measurement.

FIG. 12 shows a screenshot of the second exemplary image including a 3D overlay on a lesion.

FIG. 13 shows a screenshot of the second exemplary image including a cross-sequence of images of the lesion.

FIG. 14 shows a screenshot of the third exemplary image including a cross-sequence visualization of a finding and pre-determined answers.

### DETAILED DESCRIPTION

The exemplary embodiments may be further understood with reference to the following description and the appended drawings, wherein like elements are referred to with the same reference numerals. The exemplary embodiments relate to a system and method for workflow integration and orchestration. In particular, the exemplary embodiments describe systems and methods which direct images to be read on imaging system workstations or a PACS workstation, where contextual cues of a user may be used to determine a desired action to be taken by a system.

Advanced image processing (AIP) and automated clinical intelligence (CI) are highly useful tools for radiologists and other medical professionals. However, they suffer from at least two distinct problems. A first problem is a lack of workflow integration. In particular, AIP, because of its computational intensive nature, is typically executed from dedicated workstations that cannot be consulted without the radiologist physically switching desks, logging on, finding the relevant patient exams, selecting the appropriate applications in the AIP environment, waiting for the applications to finalize computation, recording key outcome findings (e.g., measurements), physical moving back to the routine workstations, and, finally, copying relevant information into a radiology report.

The second problem is observer variability. In particular, as is known by those of skill in the art, the detection, quantification, characterization and diagnosis of individual lesions in radiology exams is subject to substantial intra-rater variability and even more substantial inter-rater variability. These observations negatively affect the perception of radiology as an exact science, and more concretely, may downgrade the satisfaction of referring physicians with the service of the radiology department of choice.

The exemplary embodiments discussed below provide solutions to these and other problems with AIP. Thus, the exemplary embodiments address the issues of the lack of workflow integration and observer variability, which are problems that are rooted in computer technology, and solve them.

As shown in FIGS. 1 and 2, a system 100 according to an exemplary embodiment of the present disclosure determines a desired action to be taken by a user reading an image of a patient body segment. The system 100 comprises a server 102 and a user workstation 104 including, for example, a display 106 and a user interface 108, and a processor 110. The server 102 may further include a processing device 112 including various programs and/or engines such as, for example, a Digital Imaging and Communications in Medicine (DICOM) router 114, a state aggregator 116 and a workflow orchestration engine 118, along with a memory 120. Although the server 102 is shown and described as being connected to a single user workstation 104, it will be understood by those of skill in the art that the server 102 may be connected to any number of workstations 104 on which the images to be read may be reviewed by a user.

The processor 110 may be configured to execute a plurality of applications of the server 102. For example, the applications may include the functionality associated with the workflow orchestration engine 118. It should be noted that the above applications being described as an application (e.g., a program) executed by the processor 110 is only exemplary. The functionality associated with the applications may also be represented as a separate incorporated component of the user workstation 104 or may be a modular component coupled to user workstation 104, e.g., an integrated circuit with or without firmware. For example, the processor 110 may be a hardware component that comprises circuitry necessary to interpret and execute electrical signals fed into the system 100. Examples of processors include central processing units (CPUs), control units, microprocessors, etc. The circuitry may be implemented as an integrated circuit, an application specific integrated circuit (ASIC), etc. The exemplary embodiments may be implemented in any of these or other configurations of a user workstation 104.

The memory arrangement 120 may be a hardware component configured to store data related to operations performed by the workstation 104 and/or the server 102. For example, the memory arrangement 120 may store data related to the functionality of the user interface 108 or the workflow orchestration engine 118. The memory arrangement 120 may be any type of semiconductor memory, including volatile and non-volatile memory. Examples of non-volatile memory include flash memory, read only memory (ROM), programmable ROM (PROM), erasable PROM (EPROM) and electrically erasable PROM (EEPROM). Examples of volatile memory include dynamic random-access memory (DRAM), and fast CPU cache memory, which is typically static random-access memory (SRAM).

The display 106 may be a hardware component configured to show data to a user. The display 106 may be, for example, a liquid crystal display (LCD) device, a light emitting diode (LED) display, an organic LED (OLED) display, a plasma display panel (PDP), etc. Those skilled in the art will understand that the functionalities of the user interface 108 and the display 106 may be implemented in a single hardware component. For example, a touchscreen device may be used to implement both the display and the user interface 108.

The DICOM router 114 may receive images to be read, send the images to a Picture Archiving Communications System (PACS), and notify the workflow orchestration engine 118. The PACS may be a workstation that aids the user (e.g. radiologist) in their duties and allows them to keep up with ever increasing workloads. In particular, the PACS provides access to a patient study including, for example, a patient's radiological history, including diagnostic reports, exam notes, clinical history and images. Further, the PACS has features that simplify and speed up workflow to optimize the productivity of a user in reviewing volumes of patient studies in order to maintain a cost-effectiveness and efficiency.

The images received by the DICOM router 114 may, for example, be routed to both a Clinical PACS 130 and a pre-processing PACS 132. The Clinical PACS 130 stores the images to be read until a user retrieves them for reading. The pre-processing PACS 132 may, for example, identify the type of image (e.g., CT, MRI) and the body segment (e.g., lung, brain) that is imaged in an image to be read. This may help streamline the workflow process. The state aggregator 116 extracts patient information that may be pertinent to the image to be read from one or more medical repositories such as, for example, EMR (Electronic Medical Record), Radiology Information Systems, PACS, and/or clinical data feeds, through, for example, Application Programming Interfaces (APIs). The state aggregator 116 may extract information such as, for example, demographic information, active diagnoses, personal and family risk factors, prior surgery events, medications, allergies, etc. The images to be read and any corresponding information such as image type, body segment and patient information may be stored to the memory 120.

The workflow orchestration engine 118 may monitor and interpret contextual cues of a user (e.g., radiologist) reading the image using, for example, a user intention detection engine 122, AIP (Advanced Image Processing) engines 124 and CI (Clinical Intelligence) engines 126. The user intention detection engine 118 receives the contextual cues of the user and maps them to one or more user intentions such as, “acquire volumetric quantification”, “acquire anatomical location”, “acquire view of this region of interest in other sequences of this exam”, and “acquire view of this region of interest in relevant prior exam.” AIP engines 124 may include, for example, smart annotation, lesion characterization, personalized CADx and anatomical awareness engines, as shown in FIG. 2, which may provide, for example, automated lesion detection, automated lesion characterization, automated lesion segmentation, automated anatomy segmentation, automated abnormality detection and/or automated multi-sequence registration. CI engines 126 may include, for example, automated risk calculation engines, automated guidelines engines, automated diagnoses engines, automated report content generation, and automated retrieval of similar cases. The workflow orchestration engine 118 may receive a user intention object identified by the user intention detection engine 118 and initiate one or more API and/or CI engine 124, 126 via a rule-based logic, which will be described in further detail below. These rules may be stored to the memory 120.

The user workstation 104 may be a personal computing device which, as described above, includes a display 106 for viewing the image to be read and a user interface 108 which may be used for providing the contextual cues to the workflow orchestration engine 116. The user may login at the user workstation, providing user authentication information associated with a professional profile including information such as, for example, a specialty and/or seniority. The user authentication information may be used to store specific user preferences and/or to tailor the predicted desired actions to each user. In some embodiments, a user's specialty or seniority may be used to initiate specific CI engines 126 to provide decision support. For example, where a user is identified as being a junior faculty member, the server 102 may initiate an automated guideline and/or diagnosis engine to aid in providing a proper diagnosis or reading of the image to address the user's lack of experience.

The user may login and provide contextual cues using the user interface 108, which may include input devices such as, for example, a keyboard, a mouse, a touch display on the display 106 and/or an eye movement tracking device. Although the exemplary embodiments describe the contextual cues as being generated via a mouse using, it will be understood by those skilled in the art that the contextual cues may be generated in any of a variety of ways including, for example, eyeball tracking via gestures and/or eye blinks. The processor 110 communicates with the server 102 to retrieve and display images to be read, manipulate the image to be read, provide contextual cues, include findings in a radiology report, etc.

Contextual cues generated via, for example, the user interface 108 of the user workstation 104 are provided to the workflow orchestration engine 118, which receives the user intention object and orchestrates one or more AIP and/or CI engines 124, 126. As described above, the dynamic sequencing of engines can be driven by rule-based logic. Examples of rule-based logic may be as follows. If the user intention is determined to be “acquire volumetric quantification”, then retrieve the three-dimensional segmentation of the lesion that is nearest the voxel selected by the user interface 108 (e.g., mouse device). If the user intention is determined to be “annotate lesion”, then retrieve the anatomical location of the mouse device and funnel the output into the Smart Annotation CI engine. If the output of the Smart Annotation CU equals “nodule” and the anatomical location equals “lung”, then run the Lung nodule segmentation AIP engine or retrieve its output if it has already been run in pre-processing mode. If a finding was annotated “nodule” and the anatomical location equals “lung”, then retrieve smoking status and age from state aggregator 116 and feed these as input values to the follow-up recommendation engine. If a finding as annotated “nodule”, the anatomical location equals “lung” and the user intention is “determine malignancy risk”, retrieve age and smoking status from the state aggregator 116, retrieve spiculation characterization and emphysema detection information from the appropriate AIP engines 124 and feed them to the risk model. It will be understood by those of skill in the art that the above outlined rules are exemplary only, and that any number of rules may be instituted to mimic a decision/diagnosis process of a user or to follow accepted guidelines within the industry. In addition, although the exemplary embodiment specifically describes the diagnosis and reading of a lung nodule, the system and method of the present disclosure may be used for the reading/diagnoses of lesions/nodules in any of a variety of body segments and types of images.

Once the user's contextual cues have produced a predicted desired action of the user, the user may modify the results, accept the results or reject the results. Accepting or rejecting the results may indicate to the server 102 whether the workflow orchestration engine 118 properly determined/predicted the desired action of the user. Each user-system dialogue, which may include contextual cues, derived user information, state information retrieved from the state aggregator 116, image information computed by AIP engines 124, decision support computed by CI engines 126 and/or the action of the user in response to the displayed decision support, may be stored to a prior user-machines dialogues database 134 in the memory 120.

According to a further exemplary embodiment, the system 100 may further include a machine intelligence layer 128 which accesses prior user-system dialogues stored in the memory 120 to train the system 100 using machine learning techniques. The machine intelligence layer 128 may be included in the workflow orchestration engine 118, as shown in FIG. 3. The machine intelligence layer 128 may predict the positive user responses to determine whether the system 100 is properly predicting the desired actions of the user and/or providing the proper decision support to the user. For example, where a user accepts an action predicted by the workflow orchestration engine 118, the machine intelligence layer 128 would interpret the acceptance as a positive user response. If the user rejects the predicted action, the machine intelligence layer 128 may interpret the rejection as a negative user response. The workflow orchestration engine 118 may alter the predicted actions and/or provided decision support based on the positive/negative responses of the user.

Alternatively, in another embodiment, each engine (e.g., AIP engines 124, CI engines 126) may include its own machine intelligence layer, as shown in FIG. 2. This machine intelligence layer may be optimized to predict the user's feedback on the output of the individual engines based on the contextual cues and the initial output of the engine itself. When applied on a series of contextual cues and the initial output of the engine, the outcome of the machine intelligence layer may be different from the engine's output. In that case, the machine intelligence layer may overrule the original engine.

FIG. 4 shows a method 200 according to an exemplary embodiment for predicting a desired user action based on contextual cues provided by the user when reading an image. In a step 210, a DICOM router 114 directs images to be read to, for example, the Clinical PACS 130 for storage prior to being retrieved and read by a user and notifies the workflow orchestration engine 118 that the images have been stored and are ready to be read. The DICOM router 114 may also direct the images to be read to the pre-processing PACS so that the type of image and a body segment being imaged may be identified for each of the images to be read, in a step 220. In a step 230, the state aggregator 116 may also extract relevant patient information for each of the images to be read. As described above with respect to the system 100, the patient information may include, for example, demographic information, active diagnoses, personal and family risk factors, prior surgery events, medications, allergies, etc.

Once the images to be read have been processed, as described above, a user may retrieve one of the images to be read, in a step 240, so that the image to be read is displayed on the display 106. As described above, the user may login to the user workstation 104 so that the user profile, which may include authentication information, is also available to the workflow orchestration engine 118. During the reading of the image, the user may provide contextual cues to the server 102 via the user interface 108, in a step 250. The contextual cues may be continuously monitored by the workflow orchestration engine 118. In a step 260, the user intention detection engine 122 maps the detected contextual cue to a user intention. For example, when the user hovers his/her mouse pointer over a lesion in a region of interest on the displayed image, the user intention detection engine 122 may determine that the user's intention is to annotate the lesion. In another example, when the user takes a measurement of the lesion by, for example, drawing a line across a width of the lesion, the user intention detection engine 122 may determine that the user's intention is to acquire a volumetric quantification of the lesion.

Additional examples of contextual cues include zooming in or out of the image, clicking on the image, and creating a rectangular region of interest. In addition, movements of the mouse or other input device can be included as cues, with pre-determined semantics, similar to smart phone “swipe” functionality. In an advanced embodiment, if an eye-ball tracker is installed, eye movements can be included in the list of contextual cues. It will be understood by those of skill in the art that any of the contextual cues described above may be pre-assigned a corresponding user intention.

In a step 270, the workflow orchestration engine 118 generates an action based on the user intention. Where the user's intention was determined to be to annotate the lesion, an annotation of the lesion may be displayed over the image. The type of image and body segment identified in the step 220 may be used to annotate the lesion. For example, as shown in FIG. 7, where the image was identified as a CT scan of a lung, the workflow orchestration engine 118 may run, for example, a smart annotation engine of the AIP engines 124, which identifies the lesion as a nodule in the right, lower lobe of the lung and annotates the lesion as such. In another example, as shown in FIG. 8, where the user intention was determined to be to acquire a volumetric quantification of the lesion, the workflow orchestration engine 118 may run a lung nodule segmentation engine of the AIP engines 124, to overlay a 3D volume over the lesion in the displayed image, to determine the three dimensional volume of the lesion. The determined three-dimensional volume, along with any additional available characteristic information (e.g., relevant patient information, spiculation, node count) of the lesion, may be displayed on the display 106.

In another embodiment, contextual cues may also include cues that are not directly provided by the user interface 108 (e.g., mouse). For example, a seniority of the user, which may be identified during the user login, may be used to generate decision support actions. In one example, as shown in FIG. 9, where the user is identified as junior faculty, the workflow orchestration engine 118 may run a guideline engine of the CI engines 126 to launch a prediction model such as, for example, the Vancouver prediction model, which estimates malignancy for lung modules. Using previously obtained parameters such as, for example, patient information identified by the state aggregator 116 and a determined size of the lesion, the guideline engine may aid in determining a malignancy of the lesion.

In a step 280, the user may accept, reject or modify the action generated in the step 270. For example, where the annotation was correctly generated, the user may accept the displayed annotation. If the user, however, believes that the annotation was incorrect, the user may reject or modify the annotation. In another example, where the 3D volume overlaid over the lesion is believed to be correct, the user may accept the overlaid volume. If, however, the user believes that the overlaid volume is incorrect, the user may modify contours of the overlaid volume by, for example, clicking and dragging portions thereof, to generate a more accurate three-dimensional quantification of the lesion. In yet another example, where the user believes that the predicted malignancy of the lesion is incorrect, the user may reject or modify the results accordingly. As described above with respect to the system 100, the user's responses to the generated actions may be interpreted via the machine intelligence layer 128 to “train” the system and method regarding the user's desired actions with respect to images being read. Thus, during future readings of images, the system and method may be more accurately able to determine/predict the intentions of the user.

The steps 250 to 280 may be repeated, as necessary, until all of the contextual cues of the user have been interpreted to generate a corresponding predicted action. For example, the workflow orchestration engine 118 may annotate the lesion, determine a three dimensional quantification of the image and provide a predicted malignancy of the lesion during a single reading of the image. In some embodiments, the method 200 may additionally predict further actions desired by the user, in a step 290, based on prior contextual cues and/or generated actions. For example, the workflow orchestration engine 118 may predict that the user may desire to include all of the information derived during the reading of the image to a radiology report for the image. In this example, the workflow orchestration engine 118 may generate and display a sentence characterizing the lesion and providing a diagnosis, as shown in FIG. 10. The user may then copy and paste the sentence into the radiology report or, if an appropriate AIP engine is in place, the sentence may be directly exported to be included in the radiology report.

Although the method 200 is described above in regard to a CT scan of a lung nodule, it will be understood by those of skill in the art that the exemplary system and method of the present disclosure may be used during the reading of any of a variety of image types and body segments. In another example, the system 100 and method 200 may be used for the reading of a neurological MRI image. In this example, a user retrieves the image to be read such that it is displayed on the display 106 of the user workstation 104, in the step 240. This image may have been previously stored and processed to identify the type of image and body segment, as well as extract relevant patient information, during the steps 210-230. Once the image has been displayed, as shown in FIG. 11, the user may make a linear measurement in a 2D plane of a neurological lesion shown in the displayed image, in the step 250. In the step 260, this contextual cue is mapped to the user intention of acquiring a three dimensional volume of the neurological lesion. In a step 270, a 3D volume overlay is generated over the neurological lesion, as shown in FIG. 12. In the step 280, the user may accept, reject or modify the overlay, substantially as described above in regard to the lung nodule. Based on the prior contextual cues and corresponding generated actions, in the step 290, the workflow orchestration engine 118 may predict that the user is interested in inspecting the lesion cross sequence and generate a ribbon of registered slices across relevant sequences of the image, as shown in FIG. 13, to provide image context to the user.

As described above in regard to the lung nodule example, the steps 250-280 and/or 250-290 may be repeated as necessary, until all of the desired actions of the user have been generated and applied. Although the exemplary embodiments describe automatically determining/predicting desired actions of the user, it will be understood by those of skill in the art that the user may also manually request that certain actions be taken.

As shown in FIG. 5, a system 300, according to an exemplary embodiment of the present disclosure, provides contextual interpretation support suggestions to the user. The system 500 comprises the server 102 and the user workstation 104 including, for example, the display 106, the user interface 108, and the processor 110. The exemplary hardware device described above for the server 102 and the user workstation 104 may also be used in the exemplary embodiment. The server 102 may further include the processing device 112 including various programs and/or engines such as, for example, the workflow orchestration engine 118, a sequence normalization engine 130, a sequence registration engine 132, a contextual lesion interpretation support engine 134 and a controller engine 136. Although the server 102 is shown and described as being connected to a single user workstation 104, it will be understood by those of skill in the art that the server 102 may be connected to any number of workstations 104 on which the images to be read may be reviewed by a user. Further, those skilled in the art would understand the system 300 can be completely or partly integrated with the programs and/or engines of system 100.

The workflow orchestration engine 118 may provide contextual cues generated via, for example, the user interface 108 of the user workstation 104. In one example, this may be the radiologist annotating the lesion on the image. The sequence normalization engine 130 may normalize the sequences of the image (or an imaging exam) onto a controlled nomenclature of sequence names. In an exemplary embodiment, the normalization process may take into account information from a sequence header. It should be noted that the account information may be reliable if, for example, modalities at an institution are configured appropriately and/or the user has received proper training. If so, the normalization may be implemented as a table mapping sequence header names onto the controlled nomenclature of sequence names. In another exemplary embodiment, parameter settings from the DICOM router 114 may be leveraged.

The sequence registration engine 132 may register imaging slices across the sequences of at least one of a plurality of imaging exams. For example, the sequence registration engine 132 may register the imaging slices across MRI sequences of one or more distinct MRI exams. In an exemplary embodiment, the registration may be implemented in a PACS viewer, such as, for example, iSite provided by KONINKLIJKE PHILIPS ELECTRONICS N V. In a further exemplary embodiment, for more global matching across the sequences, the registration may be based on taking a whole image volume into account. In another exemplary embodiment, to obtain more localized matching across the sequences, the registration may be based on the lesion in the image.

The contextual lesion interpretation support engine 134 may control a rule base of anatomy-specific questions. The rule base may be managed, for example, by a controller entity that matches the rule base against a structured finding object to determine rules that apply. The controller entity may be, for example, the controller engine 136. When a rule applies, the user may be asked a question(s) and requested to complete the structured finding object. In an exemplary embodiment, each of the questions may be labeled as “Suggestion” or “Mandatory.” As such, the user may either be allowed to skip the question or may not have the option to skip the question. Those of skill in the art would understand that the questions may be marked in any manner desired.

In another exemplary embodiment, the rule base may be organized as a decision tree. This may be advantageous since, at any given moment, only one rule applies. In the event the rule is organized as a collection of rules, the controller engine 136 may prioritize which rule to instantiate. For example, the controller engine 136 may prioritize which of the collection of rules to instantiate by using a pre-defined rank.

Each of the questions may be determined based on answers given to previous questions. Additionally, each of the questions may be further determined by content of a structured annotation of the lesion. If, for example, information was already provided through the annotation available in the structured finding object, any questions that can be answered by the information already provided through the annotation are skipped. The instantiated rule may reflect the information desired to be present in any structured finding object, based on previously entered values. In an exemplary embodiment, the decision tree may have decision nodes. The decision nodes may be determined by, for example, contextual information, such as exam modality and anatomy.

In a further exemplary embodiment, as shown in FIG. 14, each of the questions shown to the user may be accompanied by at least one cross-sequence visualizations of a finding and at least one pre-determined answer. Here, for example, the annotated finding may be registered using the sequence registration engine 132 to generate an optimal view on an annotated area of interest.

In another exemplary embodiment, an information button may be utilized to generate background material. The background material may be indexed with, for example, the structured finding objects. Additionally, the background material may provide a presentation of relevant information. For example, the background information may display a medium sized brain tumor that is enhancing and another medium sized brain tumor that is not enhancing.

FIG. 6 shows a method 400, according to an exemplary embodiment, that provides contextual interpretation support suggestions to the user. In step 410, user interface 108 receives a contextual cue to the image from the user. As discussed above, the contextual cue may be, for example, the radiologist annotating the lesion on the image. In step 420, the sequence normalization engine 130 may normalize the sequences of the image. In step 430, the sequence registration engine 132 may register the imaging slices across the sequences of at least one of the plurality of imaging exams. In step 440, the contextual lesion interpretation support engine 134 may provide contextual lesion interpretation support suggestions to the user. As discussed above, the support suggestions may be the rule base of anatomy specific questions.

It is noted that the claims may include reference signs/numerals in accordance with PCT Rule 6.2(b). However, the present claims should not be considered to be limited to the exemplary embodiments corresponding to the reference signs/numerals.

Those skilled in the art will understand that the above-described exemplary embodiments may be implements in any number of manners, including, as a separate software module, as a combination of hardware and software, etc. For example, the state aggregator 116, the workflow orchestration engine 118, the user intention detection engine 122, the AIP engines 124, and the CI engines 126 may be programs containing lines of code that, when compiled, may be executed on a processor.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A method for determining a desired action of a user reading a medical image, comprising:
retrieving and displaying an image to be read by a user;
receiving, via a processor, a contextual cue of the user in response to the displayed image to be read;
mapping the contextual cue to a user intention via the processor; and
generating an action based on the user intention via the processor.

2. The method of claim 1, wherein the contextual cue includes one of moving a pointer over a lesion on displayed image, taking a measurement of the lesion on the displayed image, eye blinks of the user, and eye movements of the user.

3. The method of claim 1, further comprising:
identifying one of a type of image and a body segment of the image to be read and extracting patient information for the image to be read.

4. The method of claim 3, wherein the patient information includes one of demographic information, active diagnoses, personal and family risk factors, prior surgery events, medications and allergies.

5. The method of claim 1, further comprising:
receiving a user response to the generated action.

6. The method of claim 5, wherein the user response is one of accepting, rejecting and modifying the generated action.

7. The method of claim 5, further comprising training a machine learning layer of the processor based on the user response, the machine learning layer altering a further generated action based on the user response, wherein accepting the generated action indicates a positive user response, and one of rejecting and modifying the generated action indicates a negative user response.

8. The method of claim 1, further comprising:
predicting a further desired action of the user based on one of the contextual cue and the generated action.

9. The method of claim 1, wherein the contextual cue is provided by a user interface and determined via a user profile.

10. (canceled)

11. A system for determining a desired action of a user reading a medical image, comprising:
a display displaying an image to be read by a user; and
a processor receiving a contextual cue of the user in response to the displayed image to be read, mapping the contextual cue to a user intention via the processor, and generating an action based on the user intention via the processor.

12. The system of claim 11, wherein the contextual cue includes one of moving a pointer over a lesion on displayed image, taking a measurement of the lesion on the displayed image, eye blinks of the user, and eye movements of the user.

13. The system of claim 11, wherein the processor identifies one of a type of image and a body segment of the image to be read and extracts patient information for the image to be read.

14. The system of claim 11, wherein the processor receives a user response to the generated action, wherein the user response is one of accepting, rejecting and modifying the generated action.

15. (canceled)

16. The system of claim 14, wherein the processor includes a machine learning layer trained via the user response, the machine learning layer altering a further generated action based on the user response, wherein accepting the generated action indicates a positive user response, and one of rejecting and modifying the generated action indicates a negative user response.

17. The system of claim 11, wherein the processor predicts a further desired action of the user based on one of the contextual cue and the generated action.

18. (canceled)

19. (canceled)

20. (canceled)
